(19) United States
(12) Patent Application Publication
(10) Pub. No.: US 2010/0298864 A1
(43) Pub. Date: Nov. 25, 2010

(54) ARTICULATING RIGID GRASPER

(76) Inventor: Michael Castro, Fuquay Varina, NC (US)

Correspondence Address: SYNECOR LLC, P.O. BOX 5325, LARKSPUR, CA, 94977, US

(21) Appl. No.: 12/511,052

(22) Filed: Jul. 28, 2009

Related U.S. Application Data
(60) Provisional application No. 61/145,006, filed on Jan. 15, 2009.

(51) Int. Cl. A61B 17/29

(52) U.S. Cl. 606/205

(57) ABSTRACT

An articulating grasper includes an elongate shaft having an articulating distal section and a pair of jaw members on the articulating distal section. A first actuation member is coupled to the articulating distal section and is longitudinally moveable to pivot the articulating distal section. A second actuation member is coupled to at least one of the jaw members and is longitudinally moveable to pivot at least one of the jaw member between open and closed positions.

### RELATED APPLICATIONS

This application claims the benefit of U.S. Application No. 61/145,006, filed Jan. 15, 2009.

### FIELD OF THE INVENTION

The present invention relates generally to the field of surgical instruments. More particularly, the present invention relates to articulating surgical instruments.

### BACKGROUND

A surgical grasper is a conventional instrument used in open surgical procedures, as well as in less invasive procedures such as laparoscopy, single port surgery, and natural orifice procedures. A conventional grasper includes a straight rigid shaft with a pair of jaws on its distal end and a handle on the proximal end. Manipulating the working end of the conventional grasper into a desired position requires the user to pivot the entire shaft, which can be awkward particularly in procedures conducted through a small diameter access port. A grasper having an articulating distal end is more desirable; however the articulating graspers available to date have suffered from inadequate clamping forces at the jaws. The grasper described in the present application is an articulating grasper which overcomes this limitation.

### BRIEF DESCRIPTION OF THE DRAWINGS

FIG. 1A is a perspective view of an embodiment of an articulating grasper;

FIG. 1B is similar to FIG. 1A but shows the grasper in an articulated position;

FIG. 2 is a perspective view of the distal end of the grasper;

FIG. 3A is similar to FIG. 2, but shows the internal features of the grasper separate from the tubular housings;

FIG. 3B is a perspective view of the distalmost features of the grasper, with the tubular housing removed and with one of the links exploded from the assembly to show features of the tip actuation head;

FIG. 4 is a detailed view of region 4-4 identified in FIG. 3A;

FIG. 5 is a top view of the handle of the grasper;

FIG. 6 is a perspective view of the handle with the upper housing exploded from the lower housing section;

FIGS. 7A and 7B are plan views similar to FIG. 5 omitting the upper housing section of the handle. FIG. 7A shows the arrangement of the internal handle features when the jaws are opened.

FIG. 7B shows the arrangement of the internal handle features when the jaws are closed.

FIG. 8A is a perspective view of a handle employing an alternate actuation mechanism for controlling articulation of the distal section of the grasper;

FIG. 8B is similar to FIG. 8A but shows the upper handle section removed;

FIG. 8C is similar to FIG. 8B, but further shows the lever removed.

### DETAILED DESCRIPTION

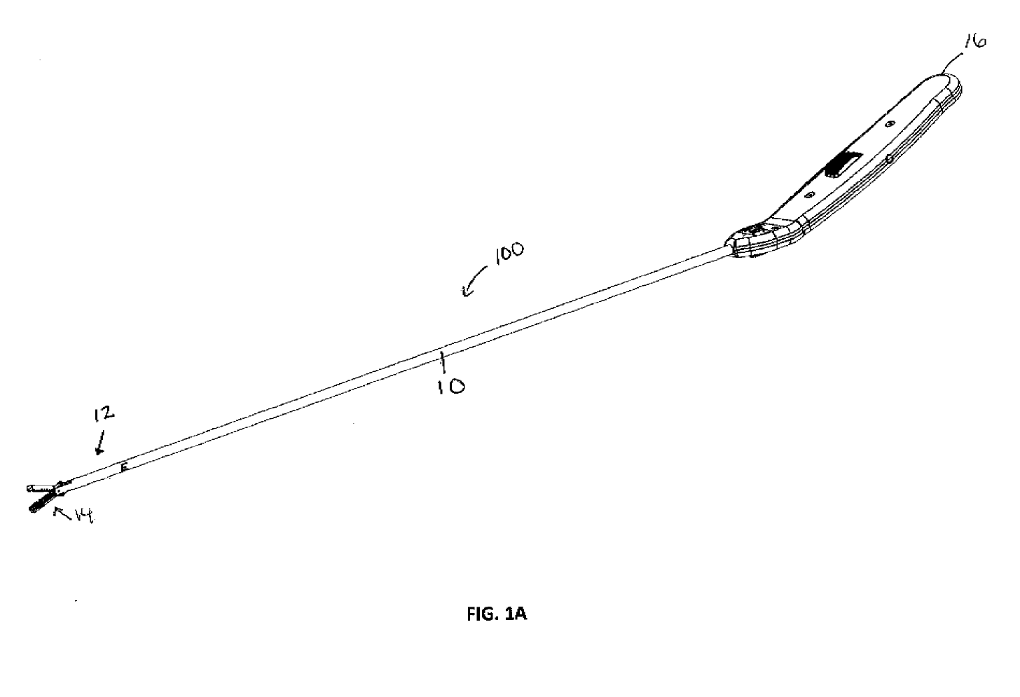

FIGS. 1A and 1B show an embodiment of an articulating grasper 100. The grasper 100 includes an elongate shaft 10 having an articulating distal section 12. Articulating section 12 is pivotable between a home position in which it is longitudinally aligned with the more proximal part of the elongate shaft 10 (FIG. 1A), and an articulated position as shown in FIG. 1B. An end effector 14, such as a pair of jaws, is positioned at the tip of the articulating section 12. A handle 16 is mounted to the proximal end of the elongate shaft 10. Handle 16 includes features for controlling articulation of the distal portion 12 and for opening/closing the jaws 14.

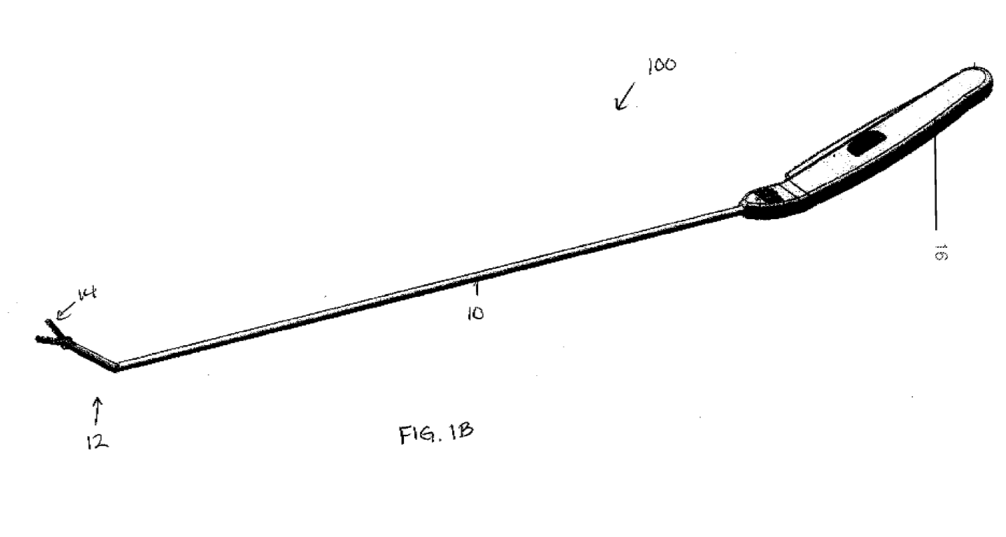

Referring to FIG. 2, the elongate shaft 10 includes a proximal tube 18 extending distally from the handle 16 (not shown). A distal tube 20 is pivotally attached to the proximal tube 18 and comprises a portion of the articulating section 12.

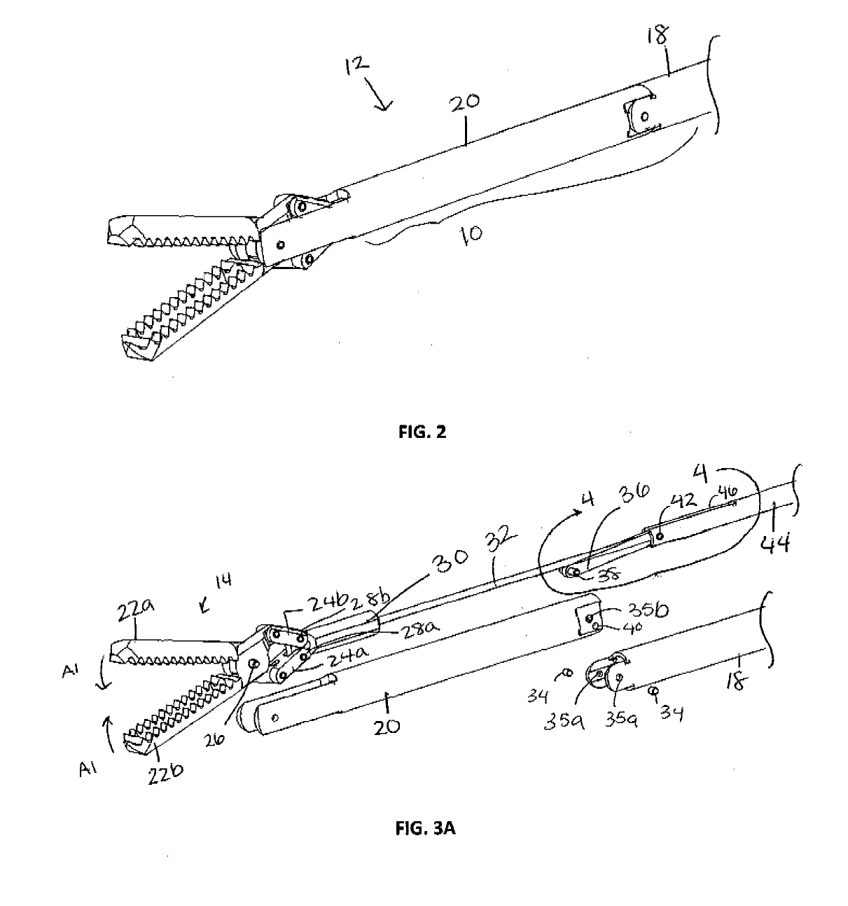

FIG. 3A is similar to FIG. 2 but shows the tubes 18, 20 removed so the components that extend through the tubes can be seen. Jaws 14 include a pair of jaw members 22a, 22b pivotable relative to a common pin 26. A pair of links 24a, 24b is coupled to the jaw members 22a, 22b. Each link 24a, 24b has a distal end pivotally attached to a corresponding one of the jaw members 22a, 22b. The proximal ends of the links 24a, 24b are pivotally attached to an actuation head 30 by pivot pins 28a, 28b. FIG. 3B shows the link 24b separated from its corresponding jaw member 22a and from the actuation head 30 to allow easy viewing of the distalmost portion of the actuation head.

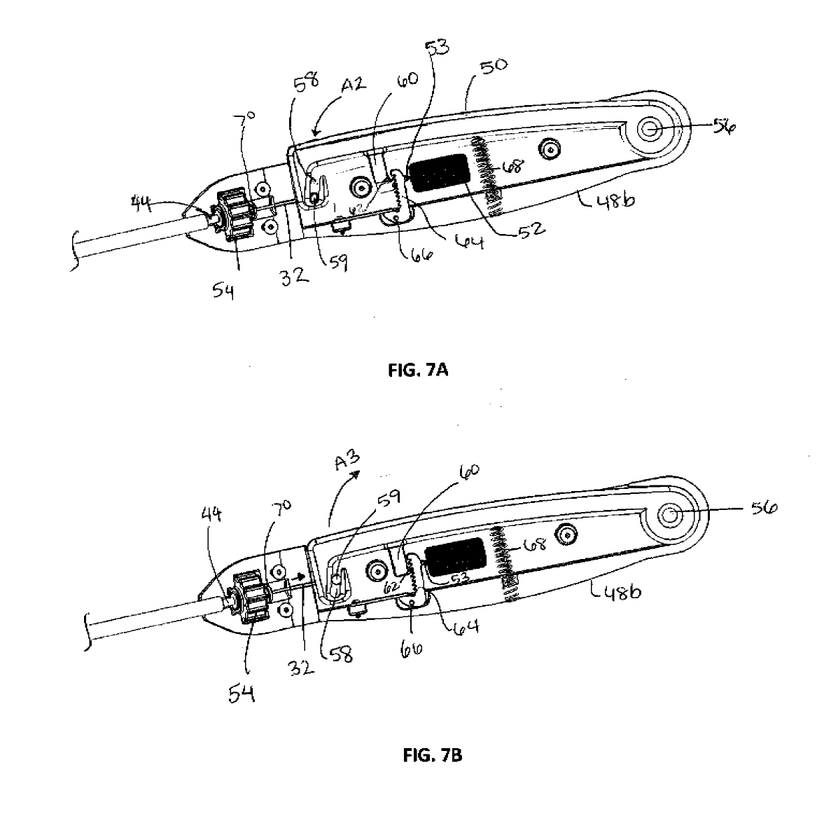

A jaw actuation member 32 extends proximally from the actuation head 30 into the handle 16 (not shown in FIGS. 3A and 3B). In use, application of tension to the jaw actuation member 32 pulls the actuation head 30 proximally, causing the distal ends of the links 24a, 24b to pivot laterally inwardly and to thus drive the jaw members 22a, 22b into the closed position as indicated by arrows A1 in FIG. 3A. In other embodiments, more than one actuation member 32 may be used. As will be discussed in detail in connection with FIG. 6, actuation features of the handle allow the user to selectively apply tension to the actuation member 32 to close the jaws of the grasper.

The jaw actuation member 32 is preferably made from a material having shape memory properties, such as a shape memory allow such as nitinol wire. In a preferred embodiment, the wire is shape set to have a straight orientation. Because of its shape memory, the jaw actuation member 32 can bend without kinking or permanent plastic deformation when the articulating section 12 is articulated relative to the longitudinal axis of the shaft. This allows clamping forces to be achieved using the jaws even when the grasper is articulated. Referring again to FIG. 3A, proximal and distal tubes 18, 20 are pivotally coupled to one another by pins 34 disposed in sidewall openings 35a, 35b in the tubes 18, 20, respectively. A link 36 is disposed within the tubes 18, 20. The distal end of the link 36 is pivotally coupled to the distal tube 20 by a pin 38 seated in sidewall opening 40 of the distal tube 20. As is evident from the relative positions of sidewall opening 35a and sidewall opening 40, the pivot axis of the pin 38 is offset from that of the pins 34. These pivot axes are preferably circumferentially offset and also longitudinally offset.

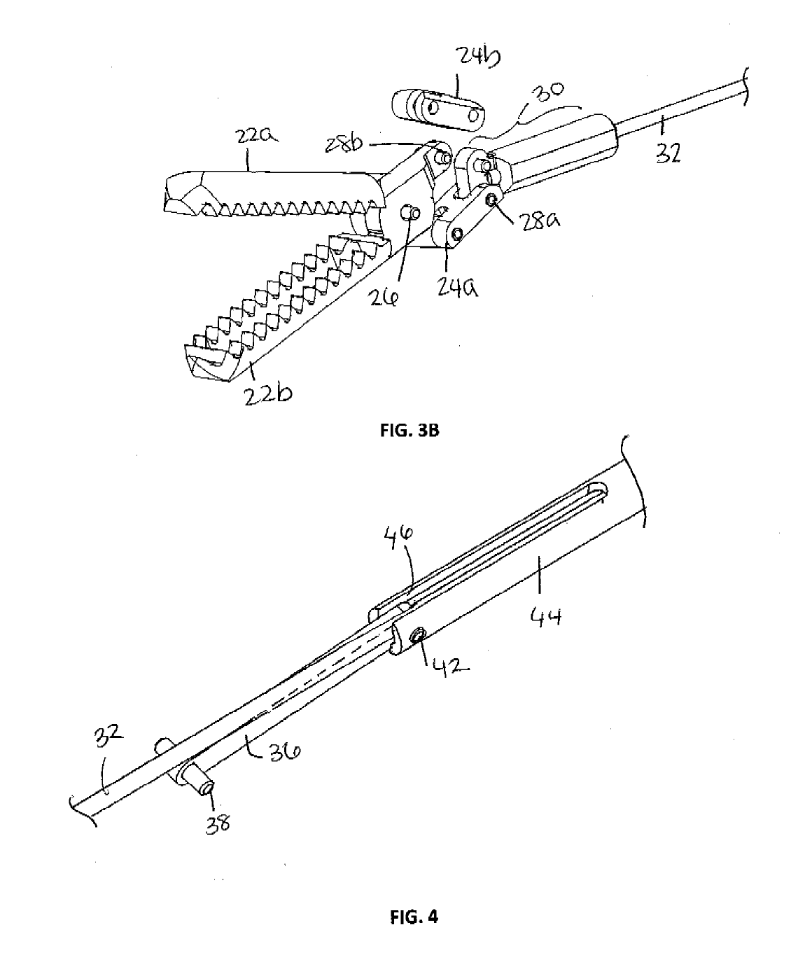

The proximal end of the link 36 is pivotally coupled, preferably by another pin 42, to a hinge actuation member 44 which extends proximally through the tube 18 into the handle 16 (not shown in FIG. 3A). In the illustrated embodiment, the hinge actuation member 44 is an elongate tube having a longitudinal slot 46 at its distal end. The proximal end of the link 36 is positioned in the slot 46 as shown. As shown in the detail view of FIG. 4, the jaw actuation member 32 is routed over the link 36 and extends into the slot 46 of the hinge actuation member 44. Tube 18 preferably includes an insert or integral component that aids in centering the hinge actuation member 44 within the tube 18 such that the two share a common longitudinal axis.

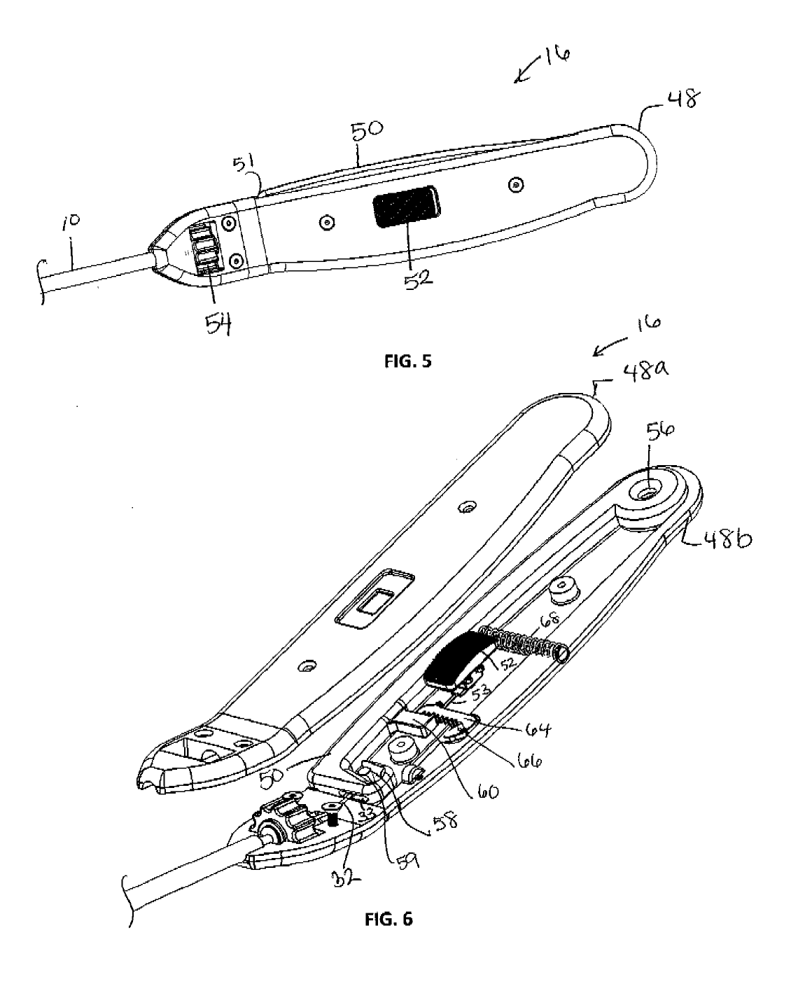

FIG. 5 is a plan view of the handle 16. The handle 16 includes a housing 48 of a size and shape suitable for holding in the palm of a user's hand. It is equipped with actuation devices allowing the user to selectively articulate the articulating section 12 (FIG. 1A) of the grasper and close/open the jaws 14 (FIG. 1A). An overview of the actuation devices will be given with reference to FIG. 5, with more detailed explanations of their features coming afterwards.

A palm actuator 50 protrudes through a slot 51 along one side of the actuator housing so as to allow the user to depress the palm actuator by squeezing the handle 16. The palm actuator 50 is used to close the jaws 14. A slider 52 is moveable in a proximal direction to release the jaws from the closed position. Articulation of the distal section 12 of the grasper is governed by knob 54.

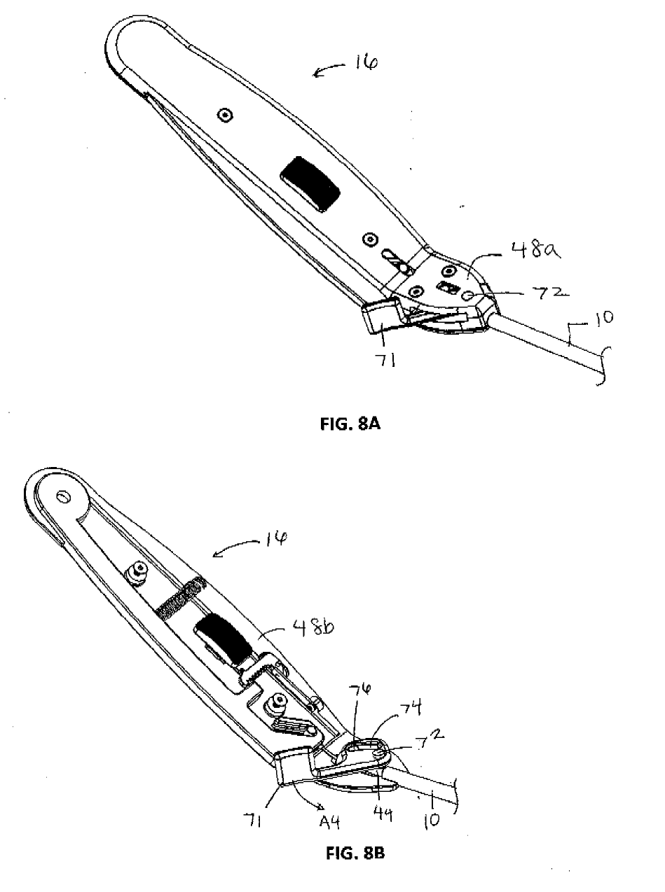

FIG. 6 shows the handle 16 with the upper housing section 48a of the housing removed from the lower housing section 48b to reveal the internal features. FIGS. 7A and 7B show only the lower housing section 48b and the internal features of the handle. Palm actuator 50 is an elongate member mounted to the lower housing section 48b at proximal pivot 56. A camming slot 58 is formed at the distal end of the palm actuator 50. A pin 59 is disposed within the camming slot. The pin 59 is attached to the proximal end of the jaw actuation member/wire 32, which extends through a slot 33 in the distal end of the palm actuator 50 to the distal end of the grasper.

A catch 60 extends laterally from the palm actuator and includes a tooth 62. A ratchet 64 is pivotally mounted to lower housing section 49b at pin 66 and positions a plurality of teeth engageable with the tooth 62 of the catch 60. Slider 52 includes a distal element 53 advanceable into contact with the ratchet 64 to cause it to pivot relative to pin 66. A spring 68 extends laterally from a side wall of the palm actuator 50 and is positioned in contact with a sidewall of the upper housing section 48a (FIG. 6).

FIGS. 7A and 7B illustrate use of the handle to operate the jaws of the grasper. When the jaws are in the open position (e.g. as in FIG. 1A), the handle components are arranged as shown in FIG. 7A, with the palm actuator 50 pivoted slightly outwardly from the housing 48 such that the pin 59 is in a distal portion of the camming slot 58.

To close the jaws, the user squeezes the handle 16 to pivot the palm actuator 50 inwardly against the spring 68 as indicated by arrow A2 in FIG. 7A. As the palm actuator 50 pivots, the camming slot 58 pushes the pin 59 to a more proximal position as shown in FIG. 7B, thereby applying tension to the jaw actuation member/wire 32 and closing the jaws as discussed above. The catch 60 moves relative to the ratchet 64 (in a downward direction relative to the orientation of FIGS. 7A and 7B), causing the ratchet 64 to pivot (relative to pin 66) against element 53. The catch 60 and the ratchet 64 re-engage to latch the jaws in the closed position.

To re-open the jaws, the user slides the slider 52 in a proximal direction to disengage the catch 60 from the ratchet 64. With the catch 60 and ratchet 64 disengaged, the palm actuator 50 is pivoted laterally outwardly as indicated by arrow A3 as the spring 68 expands to its resting position.

At the distal end of the handle 16, knob 54 is positioned on the threads of a lead screw 70 such that rotation of the knob in a first direction translates the lead screw distally, and rotation of the knob in a second direction translates the lead screw proximally. The hinge actuation member 44, first described in the discussion of FIGS. 3A and 4, is attached to the lead screw. To articulate the articulating distal section 12 of the grasper, the user rotates the knob in a first direction to translate the lead screw 70 and hinge actuation member 44 distally. To decrease the articulation, or to return the grasper to the straight configuration, the knob is rotated in an opposite direction to withdraw the lead screw 70 and hinge actuation member 44 proximally.

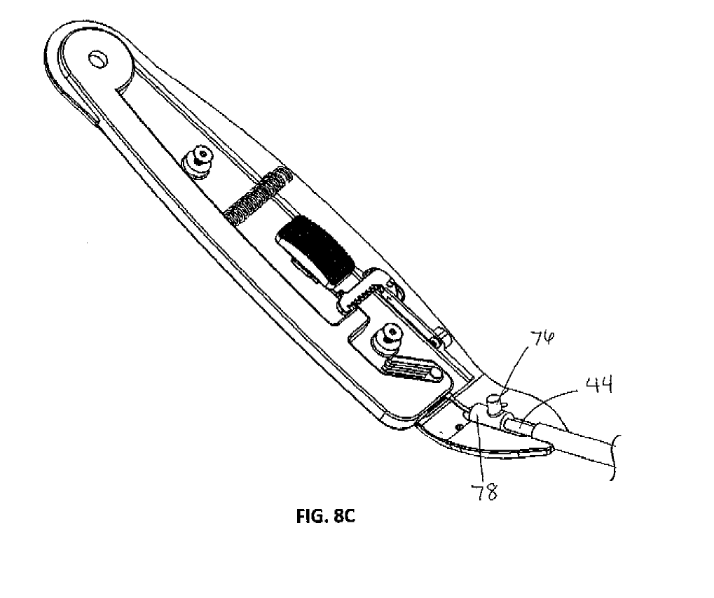

FIG. 8A shows an alternate embodiment for translating the hinge actuating member 44 using a lever 71. The lever 71 is pivotable about a pin 72 that is connected to the upper housing section 48a. As shown in FIG. 8B, the lever includes a plate having a slot 74. A pin 76 is disposed in the slot 74. FIG. 8C illustrates that the pin 76 is coupled to the hinge actuation member 44 by a tubular cap 78. To move the hinge actuation member 44 distally, the lever 71 is rotated distally relative to the pin 72 (arrow A4), thereby camming the pin 76 and thus the hinge actuation member 44 distally. The actuation member 44 is moved proximally by withdrawing the lever 71 so as to cam the pin 76 in a proximal direction.

Referring to FIG. 3A, distal movement of hinge actuation member 44 causes link 36 to pivot about the pin 42 coupling it to the hinge actuation member 44. The distal end of the link 36 moves downwardly against the proximal end of the distal tube 20, causing the distal end of the tube 20 to pivot upwardly into the articulated position shown in FIG. 1B. The user can control the degree of articulation by controlling the amount by which s/he rotates the knob 54.

The instrument may be provided with instructions for use instructing the user to operate the articulating grasper using methods described herein or equivalent thereto.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed.

Any and all patents, patent applications and printed publications referred to above, including for purposes of priority, are incorporated by reference.

1. A medical instrument comprising:
an elongate shaft having a proximal section and an articulating distal section, the distal section pivotable relative to the proximal section between a first position and a second position;
a pair of jaw members on the articulating distal section, at least one of the jaw members pivotable relative to the other jaw member;
a first actuation member coupled to the articulating distal section, the first actuation member longitudinally moveable relative to the proximal section of the elongate shaft to pivot the articulating distal section between the first and second positions; and
a second actuation member coupled to at least one of the jaw members, the second actuation member longitudinally moveable relative to the elongate shaft to pivot the at least one jaw member between open and closed positions.

2. The medical instrument of claim 1, wherein the elongate shaft is a tubular shaft, and wherein the second actuation member extends at least partially through the articulating distal section and the proximal section of the tubular shaft.

3. The medical instrument of claim 2, wherein the first actuation member includes a tubular member extending partially through the elongate shaft and wherein the second actuation member extends longitudinally through the first actuation member.

4. The medical instrument of claim 3, further including a handle coupled to the proximal section of the elongate shaft, the handle including
a first actuator operatively associated with the first actuation member, the first actuator manipulatable by a user to longitudinally move the first actuation member relative to the proximal section of the elongate shaft to articulate the articulating distal section; and
a second actuator operatively associated with the second actuation member, the second actuator manipulatable by a user to longitudinally move the second actuation member relative to the elongate shaft to pivot the at least one jaw member.

5. The medical instrument of claim 1, wherein in the first position the distal section extends longitudinally from the proximal section, and wherein in the second position the distal section extends angularly from the proximal section.

6. The medical instrument of claim 1, wherein the second actuation member is formed of a shape memory material.

7. The medical instrument of claim 1, wherein the second actuation member is formed of a nickel titanium alloy.

8. The medical instrument of claim 1, wherein the distal section is pivotally attached to the proximal section.

9. The medical instrument of claim 8 wherein the first actuation member includes a distal link having a distal end pivotally coupled to the articulating distal section, and a proximal member pivotally coupled to the distal link, the distal link pivotable relative to the proximal member between first and second positions in response to longitudinal movement of the proximal member relative to the elongate shaft.

10. The medical instrument of claim 9, wherein the proximal member is a tubular proximal member having a lumen, and wherein the second actuating member is longitudinally slidable within the lumen of the tubular proximal member.

11. The medical instrument of claim 10, wherein the tubular proximal member includes a slot, and wherein the second actuating member extends through the slot into the lumen.

12. The medical instrument of claim 1, wherein the second actuating member is coupled to each of the jaw members, such that longitudinal movement of the second actuating member in a proximal direction opens the jaw members.

13. A method of using a medical instrument, comprising:
providing a medical instrument including an elongate shaft having a proximal section and an articulating distal section pivotally coupled to the proximal section, and further including jaw members mounted to the distal section;
pivoting the articulating distal section into an articulated position relative to the proximal section; and
opening and closing the jaws when the articulating distal section is in the articulated position.

14. The method of claim 13, wherein the elongate shaft is a tubular shaft, and wherein the medical instrument includes a jaw actuation member extending through the proximal section and the articulating distal section of the elongate shaft, and wherein closing the jaws includes moving the jaw actuation member in a longitudinal direction within the elongate shaft.

15. The method of claim 14, wherein pivoting the articulating distal section bends the jaw actuation member from a first position to a second position but does not result in plastic deformation of the jaw actuation member.

16. The method of claim 14, wherein:
providing the medical instrument provides the instrument to include an articulation actuation member, the articulation actuation member including:
a distal link disposed within the elongate shaft, the distal link having a distal end pivotally coupled to the articulating distal section; and
a proximal tube disposed within the elongate shaft, the proximal tube pivotally coupled to the distal link; and
pivoting the articulating distal section includes sliding the proximal tube longitudinally within the elongate shaft to pivot the distal link.
